(19) United States
(12) Patent Application Publication
(10) Pub. No.: US 2025/0303232 A1
(43) Pub. Date: Oct. 2, 2025

(54) BIOLOGY BASED FITNESS APPLICATION

(71) Applicant: Lucas Garcia, Chino Hills, CA (US)

(72) Inventor: Lucas Garcia, Chino Hills, CA (US)

(21) Appl. No.: 19/087,532

(22) Filed: Mar. 23, 2025

Related U.S. Application Data
(60) Provisional application No. 63/573,187, filed on Apr. 2, 2024.

(51) Int. Cl. A63B 24/00; G16H 20/30; G16H 20/60

(52) U.S. Cl. CPC A63B 24/0075; G16H 20/30; G16H 20/60

(57) ABSTRACT

A fitness application is described. The fitness application has a graphical user interface that can input or receive one or more parameters of a user or a fitness or health regimen and provide custom functionalities such as scheduling, monitoring, tracking, and suggesting/recommending various health regimens specified for individuals based on the input data.

### CROSS-REFERENCE TO RELATED APPLICATIONS

The present application relies on the disclosure of and claims priority to and the benefit of the filing date of U.S. Provisional Application No. 63/573,187, filed Apr. 2, 2024, the disclosure of which is hereby incorporated by reference herein in its entirety.

### FIELD

The present disclosure is directed to the field of fitness applications. More particularly, the present disclosure is directed to fitness applications with a graphical user interface that can input or receive one or more parameters of a user or a fitness or health regimen and provide custom functionalities such as scheduling, monitoring, tracking, and suggesting/recommending various health regimens specified for individuals based on the input data.

### DETAILED DESCRIPTION

Reference will now be made in detail to various illustrative implementations. It is to be understood that the following discussion of the implementations is not intended to be limiting.

The present disclosure provides a biology fitness program graphical user interface or user program, also referred to herein as an application. The graphical user interface allows one or more parameters of a fitness or health regimen to be inputted or entered on the user interface by a user, as demonstrated in the User Interface example below. The application provides custom functionalities such as scheduling, monitoring, tracking, and suggesting/recommending various health regimens specified for individuals based on input data received at the start of the program from the particular user. The user interface is designed for input of one or more parameters including user parameters such as height, weight, age, gender at birth, body fat percentage, waist size, hip size, shoulder size, chest size, neck circumference, bicep circumference, leg length, and arm length. The user interface also allows input of one or more parameters of an exercise regimen including the particular exercise equipment or method, the muscles targeted, speed, power, length of exercise, weight or resistance, number of repetitions, number of sets, length of breaks between sets or activities, and workout schedule. The particular exercise equipment entered can include a rower or ergometer, stationary bike, elliptical machine, treadmill, cross country skiing machine, weight training machine, other fitness machine, or free weights. The particular exercise method entered can include walking, running, swimming, biking, skating, rollerblading, jump roping, boxing, various calisthenics such as pushups, chin-ups, and squats, or various stretches. The muscles targeted can include biceps, triceps, quadrilaterals, abdominals, shoulders, back, lower body including legs, calves, and ankles, upper body including arms, as well as core muscles. Various exercises of the health regimens can include bench press, shoulder press, bicep curls, tricep curls, and lateral pull downs, to name a few; other exercises are detailed in the Examples below. The user interface can also provide input of nutritional information such as calories, macronutrients (protein, fat, carbohydrates), micronutrients (the vitamins and minerals), food groups (meat, dairy, fruits, vegetables, complex carbohydrates), specific types of diets (vegan, vegetarian, carnivore, Mediterranean, keto) or nutritional supplements such as types, amounts, portions, and/or consumption schedules, frequencies, or details of the nutrients or supplements such as time of day, amount, and whether to take with a meal or before or after a workout. The user interface can also provide input of active pharmacological ingredients such as caffeine including amount and consumption schedule. The user interface can also provide input of other health regimens such as sauna, ice baths, massage, tai chi, Pilates, yoga, and dance classes. The user interface can also provide input of other health regimens such as various martial arts such as karate, taekwondo, and jujitsu. The user interface can also provide input of other health regimens such as various sports including racquetball, paddleball, pickleball, and tennis. The user interface can also include input of various mental challenges or workouts such as crossword puzzles, Sudoku, Wordle, or chess. These are merely illustrative examples of the user input provided by the graphical user interface and additional examples will be apparent from the foregoing examples below.

The user interface can be included as part of a health or fitness program or application that is designed to schedule, monitor, track, and/or suggest or recommend various health regimens for a user based on the inputted parameters for the particular individual user. The scheduling, monitoring, tracking, and suggesting/recommending functions can encompass any parameter of a health regimen disclosed herein. The schedule functions can include a calendar that populates dates and times with the various parameters of the health regimens that facilitate execution of the various health regimens. These parameters can include various workouts such as those provided in the Examples below. The parameters can also include other aspects of health regimens such as nutritional supplement intake, examples of which are provided below, or any of the other parameters discussed herein. The monitoring and tracking functions can include input or output that tracks or monitors the achievements of various health or fitness goals, such as weight or body fat loss or gains of strength or muscle mass. The monitoring and tracking functions can also include graphical plots of various monitored parameters of health regimens including the aforementioned achievements. The suggesting/recommending functions can include output of suggested workouts or programs that can be implemented by the user such as the aforementioned exercise or dietary regimens. The suggested workouts or programs can be scheduled according to the schedule functions and tracked or monitored according to the tracking functions. The suggested health regimen programs can also include an explanation for the rationale for the particularly health regimen and the expected physiological and biological effects and benefits. As such, the application can have any combination of functionalities chosen from scheduling, monitoring, tracking, and suggesting/recommending various health regimens for a user.

The application can be used in combination with various digital health or fitness gear including step trackers, heart rate monitors, breath monitors, sleep monitors, blood pressure monitors, pulse oximeters, digital scales, glucose monitors, and ketone monitors. Such trackers and monitors can provide input to the application directly and factor into the various scheduling, monitoring, tracking, and suggesting/recommending functions of the application. Alternatively or in addition, the application can receive input from various custom applications built for the trackers and monitors, or other health applications such as various dietary tracking applications. The application can also be used in combination with various fitness equipment and gear by receiving digital input of a record of the user's performance using the fitness equipment and gear, and such input can also factor into the various functionalities of the application.

The user interface can be implemented as one or more graphics or icons including one or more data entry fields, lists with check boxes, pull-downs, or toggle switches which allow the user to input the various parameters of the health regimen. The user interface can also be organized into tabs according to the type of health regimen, each tab linked to a separate screen for entering the particular parameters of the health regimen. The user interface can alternatively create pop-up windows for each type of health regimen after selection on a screen. Inputting steps can be performed through traditional inputting means such as a physical keyboard or a keyboard projected on a display, such as a touchscreen. The keyboard can be a QWERTY keyboard or a condensed keyboard commonly used in electronic devices such as cell phones. The user interface and scheduling, monitoring, tracking, and/or suggesting or recommending functions can be implemented as computer-readable code providing one or more instructions or software using computer programming languages known in the art such as PHP, HTML, JavaScript, C, C#, C++, Java, Python, Perl, Ruby, Swift, Visual Basic, and Objective C. By such programming, the computer-readable code, computer-readable instructions, computer-executable instructions, or “software” instruct one or more processors of a computer or computing device to carry out the operations and commands of the application including the display of the various icons providing input on the graphical user interface as well as the scheduling, monitoring, tracking, and/or suggesting or recommending functions. The computer-executable code or instructions can be configured to be downloadable onto the memory or memories of the computer(s) or computing device(s) from a cloud storage source available on the Internet, such as an application retail source (e.g., “app store”) capable of being accessed from a mobile phone, tablet, desktop computer, or other programmable device having components or features capable of performing the method steps. Examples of suitable Internet-accessible sources include the Apple Store, Google Play, and other sites that make software applications and other downloads available for purchase or license.

The user interfaces can be downloaded together as an application from cloud storage services providing retail application downloading to the user's computer or computing device for hosting on the user's computer or computing device, or can be hosted on a remote computer which acts as a web server and is accessed through the Internet through an Internet browser on the user's computer or computing device. The user's computer or computing device or the remote computer(s) can receive input of one or more parameters of a fitness or health regimen by the user interface (either provided as the application on the user's computer or device, or accessed through the web server) and can have a memory capable of housing storage of the one or more parameters. The parameters can be stored in relational databases (e.g., Oracle, SQL Server) stored in memory that communicate by way of a database server or servers with the user's computer or computing device or other remote computers, such as one or more servers. The remote computer(s) can include a set of computer-executable instructions stored in memory which can be used to perform functions based on the stored information, such as scheduling, monitoring, tracking, and suggesting or recommending various health regimens; alternatively, or in addition, the user's computer or computing device can have the computer-executable instructions for performing the functions stored in memory.

The computer and/or computing device have a memory for storing the software. The memory can be implemented through non-transitory computer-readable storage media such as RAM. As used in the context of this specification, a “non-transitory computer-readable storage medium (or media)” may include any kind of computer memory, including magnetic storage media, optical storage media, nonvolatile memory storage media, and volatile memory. Non-limiting examples of non-transitory computer-readable storage media include floppy disks, magnetic tape, conventional hard disks, CD-ROM, DVD-ROM, BLU-RAY, Flash ROM, memory cards, optical drives, solid state drives, flash drives, erasable programmable read only memory (EPROM), electrically erasable programmable read-only memory (EEPROM), non-volatile ROM, and RAM.

Computers, computing devices, or systems thereof can implement the application by way of one or more processors that execute the computer-executable instructions. The computer, computing device, or system or combination thereof can include one or more stand-alone computer, such as a desktop computer, a portable computer, such as a tablet, laptop, PDA, or smartphone, or a set of computers or devices connected through a network including a client-server configuration and one or more database servers. The network can use any suitable network protocol, including IP, UDP, or ICMP, and may be any suitable wired or wireless network including any local area network, wide area network, Internet network, telecommunications network, Wi-Fi enabled network, or Bluetooth enabled network. The computers, computing devices, or systems thereof can include a variety of components known in the art, including one or more processor, a volatile memory, a non-volatile memory, standard I/O interfaces such as a universal serial bus (USB) port, an HDMI or HDMI ARC port, an optical port, an ethernet port, and/or a serial port, a hard drive, a disk drive, a CD-ROM drive, a motherboard, a printed circuit board (PCB), circuitry for enabling a wireless connection or communication to another device, such as a BLUETOOTH® board, a Wi-Fi board, or a transmitter-receiver for mobile telecommunications, a data bus, an address bus, a control bus, and/or one or more user interface devices including a display, keyboard, keypad, trackpad, mouse, control panel, touch screen display, speaker, camera, and/or microphone. The computers, computing devices, or systems thereof can be equipped with an operating system implemented as software or firmware.

The following Examples further illustrate the capabilities of the application with respect to the types of information capable of being inputted by way of the graphical user interface of the application as well as the application's various functionalities. Example 1 provides an example of the information entered into the User Interface at the start of the program. Example 2 provides an example for a user who is a woman over age 50. Example 3 provides an example for a user who is a male athlete.

### Example 1: User Interface

Enter height weight age one rep max squat deadlift bench hang clean/power clean shoulder press lateral raise front raise strict curl/bicep curl

Also enter Vo2 max and 5 mile run time optional 1 mile rower time max speed and power rower time how long you can hold top speed and power optional 5 mile stationary bike/assault bike time max speed and power stationary bike/assault bike how long you can hold top speed and power optional 1 mile vertimax time max speed and power vertimax how long you can hold top speed and power optional max speed and power jump rope how long you can hold top speed and power

Also enter goal to build a lot of muscle mass, gain size, lose fat/be leaner be more athletic, be more in shape, be stronger choose one or more or all that apply

It will range the difference between set breaks and computer based on answer will generate the workout completely

It will choose workout days muscle group depending on answer and how much general muscles get worked out if goal is to get leaner or more athletic for example don't want to gain size probably minimal amount of actual weight training days only 1 day per body region

Be in competitive environment around men fight etc don't be around babies
challenge body and maximal strength/joint strength and maximal strength each week do extremely hard movement/movements one rep max or push yourself in position body can't push so it's just pushing against resistance like a wall or so

### High Protein

Sprint 2-3 times a week not to much because will over work central nervous system

It takes 24 all the way up to 72 hours to recover from a day of sprinting

Choose Sprint Days 2-3 Times a week Monday-Saturday

Choose Back Day 1-3 times a week Monday-Saturday

Choose Ab Day 1-3 times a week Monday-Saturday

Choose Upper Body Day 1-3 times a week Monday-Saturday

Choose Lower body day 1-3 times a week Monday-Saturday

2-5 minute break after every sprint

Abs 1-3 times a week to keep core strong

### Increase Testosterone Release Estrogen

Gym to field transfer explosive gym movements to field/court to your sport

After abs laugh by watching comedy something funny this lowers cortisol increases health and flexes abs while laughing making stronger core

### Cold Bath for Testosterone or Cryo Therapy

Choose your set breaks and how long you want between 20 seconds to 5 minutes break you choose your breaks between sets depending on goal also program can generate program depending on goal of strength, hypertrophy, muscle endurance mixture of a couple or all the options will generate the set breaks or you can choose set breaks however you want

Have to choose at least one 10 minute break in each workout you get to choose you get between 1-3 10 minute breaks per workout except for in stretching no 10 minute break on explosiveness unless program says

### Sauna

reduce artificial dopamine

Mentally challenge early in morning

Mentally challenge yourself each day

Caffeine 90-120 minutes after wake up

Caffeine if you want after or around evening or after Mentally exhausting thing

Cold exposure 1-3 minutes a day usually recommended 3 minutes

Physically challenge one thing each day

Reduce sugar

sunlight when wake up

Protein drink or food with fish oil in morning

Reduce light very dark about a hour or so before bed

At least 11 minutes cold exposure every week

Ice bath before workout or way after workout to not decrease hypertrophy 1-3 minutes

Sauna 10-20 minutes

Sauna 1-6 times a week although can do daily if you feel good doing it and safe for you to do so

Ice bath 1-7 times a week keep in mind narrows blood vessels and decrease blood flow to muscles so use safely

One rep max week last month of the year of program when you go for one rep max go 10% above normal after working set after last of working sets 1-3 reps only since going for one rep max till you get to 100% of your one rep max then go for your new one rep max however many sets it takes go as feel comfortable until you can max out only 1 rep

### Monk Mode

Sprint days 1-3 times a week only do 1 explosiveness day

### For Ultra Version

Ashwaganda Tongkat Ali Tribulus Fabiola Agrestis Fish Oil Black Label Supplements Pure Creatine George's St Pierre Warrior Liver Supplement Grass Fed Beef Liver Capsules

### Chemix Gorilla Mind Gorilla Mode

Ashwaganda

250-1,000 mg daily 1-3 capsules daily

Organic Ashwaganda 2,100 mg 100 vegan capsules recommend take in morning evening any time or evening about 30 minutes to a hour after meal or before night 30 minutes to a hour before bed and some recommendations 30 minutes to at most two hours before bed

Tongkat Ali

100-300 mg daily 1 capsule daily

Now Foods Vegetarian TestoJacj Men's Support Formula, 60 ct

recommend take in morning 7-8 in morning or between meals or a hour before a workout or super hard workout it can be taken any time of day although it is a natural steroid and testosterone boosting supplement it can also significantly improve energy so can be used as a great pre workout

Tribulus Terrestris Amount 250-1,500 mg daily 1-3 capsules daily

Nutricost Tribulus Terrestris Extract recommend take 30 minutes after meal empty stomach some recommended take with water 8-12 ounces some recommended take Tribulus before workout for its anabolic effects

### Fadogia Agrestis

100-600 mg daily 1-2 capsules daily

recommend take with or without meal any time of day

### Omega 3 Fish Oil

1,000-3,000 mg daily 1-3 capsules daily

recommend take in the morning recommend take at night recommended take with meal but can also take without also recommended take right before meal you can take any time of day

### Previous Disclosure

### Biology Fitness Program User Interface User Program

Enter height weight age one rep max squat deadlift bench hang clean/power clean shoulder press lateral raise front raise strict curl/bicep curl

Also enter goal to build a lot of muscle mass, gain size, lose fat/be leaner be more athletic, be more in shape, be stronger choose one or more or all that apply

It will range the difference between set breaks and computer based on answer will generate the workout completely

It will choose workout days muscle group depending on answer and how much general muscles get worked out if goal is to get leaner or more athletic for example don't want to gain size probably minimal amount of actual weight training days only 1 day per body region

Be in competitive environment around men fight etc don't be around babies challenge body and maximal strength/joint strength and maximal strength each week do extremely hard movement/movements one rep max or push yourself in position body can't push so it's just pushing against resistance like a wall or so

### High Protein

Drink water in morning helps cognitive brain function health and physical performance

Increase testosterone release estrogen if your estrogen levels are high as a man no matter how high your testosterone is you will have all the symptoms of low testosterone

### Cold Bath for Testosterone or Cryo Therapy

Sauna can improve circulation lower blood pressure can decrease risk of heart attack sauna can decrease stress by reducing cortisol by 10-40% sauna can improve skin health reduce joint stiffness and muscle soreness can increase immune system strength also can help your muscles build bigger and stronger after workout

reduce artificial dopamine it can regulate your dopamine levels and regain brain sensitivity to natural rewards also decrease risk of mental health disorders and addictive behavior

Mentally challenge early in morning right after get sunlight 30-90 minutes sets up your brain and body especially your brain's cognitive function for rest of day it also causes neurological and biological benefits

Mentally challenge yourself each day 30-90 minutes sets up your brain and body especially your brain's cognitive function it also causes neurological and biological benefits

Caffeine 90-120 minutes after wake up wait because it allows adenosine to be cleared out and doesn't help caffeine that early because it just blocks out

Caffeine if you want after or around evening or after Mentally exhausting thing right after mentally exhausting thing 90-120 minutes after you wake up

Cold exposure 1-3 minutes a day usually recommended 3 minutes ice bath right after mentally challenging thing increases metabolism can aid recovery help neurological benefits improve insulin sensitivity increase energy levels helps your mood and immune system

Physically challenge one thing each day you will gain bone health and better strength and power for your size which will make you healthier it will increase mental and physical health

Reduce sugar help your sugar levels stay lower and help cholesterol and lowers body weight

sunlight 10-15 minutes when wake up do first thing when you wake up increase melatonin for better sleep later on and increase mood and energy for day and increases metabolism and regulates circadian rhythm and releases serotonin and increase energy and offset cortisol

Protein drink or food with fish oil in morning

Reduce light very dark about a hour or so before bed it helps better sleep cycle reduce stress and better for sleep

At least 11 minutes cold exposure every week increases metabolism can aid recovery help neurological benefits improve insulin sensitivity increase energy levels helps your mood and immune system

Ice bath before workout or way after workout to not decrease hypertrophy 1-3 minutes

Sauna 10-20 minutes

Sauna 1-6 times a week although can do daily if you feel good doing it and safe for you to do so

Ice bath 1-7 times a week keep in mind narrows blood vessels and decrease blood flow to muscles so use safely

### Monk Mode

Guaranteed for normal version of program 200-600 point increase in testosterone and at least 5 pounds of muscle gained and lose at least 5 pounds of fat and for ultra version of program 400-1000 point increase in testosterone and at least 10 pounds of muscle gained and lose at least 10 pounds of fat

### For Ultra Version

Ashwaganda Tongkat Ali Tribulus Fabiola Agrestis Fish Oil Black Label Supplements Pure Creatine George's St Pierre Warrior Liver Supplement Grass Fed Beef Liver Capsules

### Chemix Gorilla Mind Gorilla Mode

Ashwaganda

250-1,000 mg daily 1-3 capsules daily

Organic Ashwaganda 2,100 mg 100 vegan capsules

recommend take in morning evening any time or evening about 30 minutes to a hour after meal or before night 30 minutes to a hour before bed and some recommendations 30 minutes to at most two hours before bed

### Tongkat Ali

100-300 mg daily 1 capsule daily

Now Foods Vegetarian TestoJacj Men's Support Formula, 60 ct recommend take in morning 7-8 in morning or between meals or a hour before a workout or super hard workout it can be taken any time of day although it is a natural steroid and testosterone boosting supplement it can also significantly improve energy so can be used as a great pre workout

Tribulus Terrestris Amount 250-1,500 mg daily 1-3 capsules daily

Nutricost Tribulus Terrestris Extract recommend take 30 minutes after meal empty stomach some recommended take with water 8-12 ounces some recommended take Tribulus before workout for its anabolic effects

### Fadogia Agrestis

100-600 mg daily 1-2 capsules daily

recommend take with or without meal any time of day

### Omega 3 Fish Oil

1,000-3,000 mg daily 1-3 capsules daily

recommend take in the morning recommend take at night recommended take with meal but can also take without also recommended take right before meal you can take any time of day

### Creatine

5,000 mg daily 1 scoop daily recommend take after workout

recommend take before workout

recommend take in morning

recommend take with meal

Georges St Pierre Warrior Liver Supplement 6 capsules daily

recommend take with meal

recommend take after workout

recommend take before workout also

### Grass Fed Beef Liver Capsules

4-6 capsules daily

recommend take with meal

recommend take in the morning

recommend take after workout

recommend take before workout

### Chemix

1-2 scoops daily

recommend take 20-30 minutes before workout

### Gorilla Mind Gorilla Mode

1-2 scoops daily

recommend take 30 minutes to a hour before workout

recommend take right before workout

For fighter program v02 max is extremely important because in ring/cage stamina

Also enter Vo2 max and 5 mile run time optional 1 mile rower time max speed and power rower time how long you can hold top speed and power optional 5 mile stationary bike/assault bike time max speed and power stationary bike/assault bike how long you can hold top speed and power optional 1 mile vertimax time max speed and power vertimax how long you can hold top speed and power optional max speed and power jump rope how long you can hold top speed and power

### Example 2: Woman, Low End 50 Plus

### Athlete Program

10 minutes direct sunlight after waking up

30 minutes to a hour mentally challenging thing in morning

3 minute cold plunge after

1 hour stretching

15 second reach down touch toes 15 second Chinese splitz hold 15 second reach right leg half split position 15 second reach left leg half split position 15 second reach down middle split position 15 second splitz 15 second sitting half split position right leg 15 second sitting half split position left leg 15 seconds reach forward touch toes 15 second butterfly 15 second hip stretch right leg 15 second hip stretch left leg 15 second knee to chest right leg 15 second knee to chest left leg 15 second flamingo left leg 15 second flamingo right leg 10 leg swings right leg 10 leg swings left leg 20 second knee swivel left 20 second knee swivel right 20 second hip swivel left 20 second hip swivel right 20 second wrist swivel left 20 second wrist swivel right each hand 20 second elbow swivel right 20 second elbow swivel left each arm 20 second shoulder swivel left 20 second shoulder swivel right each arm back to floor on knee stretch 20 seconds 15 seconds arm behind back right arm 15 seconds arm behind back left arm 15 second leg on chair hamstring stretch left leg 15 second leg on chair hamstring stretch right leg 15 second worlds greatest left side 15 second worlds greatest right side 15 seconds leg to side on chair left leg 15 seconds leg side on chair right leg leg swings 10 reps left leg leg swings 10 reps right leg single leg rdl 10 reps left leg single leg rdl 10 reps right leg Warrior 1 15 seconds left leg Warrior 1 15 seconds right leg warrior 2 15 seconds left leg Warrior 2 15 seconds right leg balance on one leg hands forward body leaning forward yoga position 15 seconds left leg balance on one leg hands forward body leaning forward yoga position 15 seconds right leg 15 seconds hip floor wipers 15 second frog hip stretch 15 second Chinese monk squat 20 second ankle rotation left 20 second ankle rotation right each ankle alphabet each ankle 10 second plantar flex 10 second dorsi flex with ankle weight each ankle same thing without weight repeat 10 times calf stretch 10 seconds each leg for 5 sets 10 sets 10 reps calf raises leg extension with weight for 45 second hold for 10 reps same without weight leg extension hold 10 seconds for 10 reps for 5 sets repeat for 1 hour except ankle exercises repeat two times full stretch routine 30 20 second break between every stretch main stretch routine 10 second break between each stretch ankle routine

### 30 Minute Ankle Exercises

Recovery 1 hour a day 10 second each part of calf for 5 sets each leg same for 4 sides a calf 10 seconds for 5 sets and do massage gun for 1 hour or and cryotherapy or thermatech

Ice bath way before workout to not decrease hypertrophy

Sauna after workout to increase hypertrophy

At least 11 minute cold exposure each week

challenge body and maximal strength/joint strength and maximal strength each week do extremely hard movement/movements one rep max or push yourself in position body can't push so it's just pushing against resistance like a wall or so

challenge body and maximal strength/joint strength and maximal strength each week do extremely hard movement/movements one rep max or push yourself in position body can't push so it's just pushing against resistance like a wall or so

Sprint 1-2 times a week

Have to choose at least one 10 minute break in each workout you get to choose you get between 1-3 10 minute breaks per workout except for in stretching no 10 minute break on explosiveness unless program says

Reduce artificial dopamine

### Monk Mode

Mentally challenge each day

Caffeine 90-120 minutes after wake up

Caffeine if you want after or around evening or after Mentally exhausting thing

Reduce light very dark about a hour or so before bed

### Upper Body Day

10 pound dumbbells minimum for 10 reps for 2 sets each arm 10% of body weight and one rep max average barbell curls 12 reps for 2 sets 10% of body weight and one rep max average Hammer curls 10 reps for 2 sets each arm 40% of body weight and one rep max bench press 20 reps for 5 sets 40% of body weight and one rep max bench press 12 reps 40% of body weight and one rep max 8 reps for 2 sets 40% of body weight and one rep max 6 reps for 2 sets 80-85% of one rep max for 10 reps for 10 sets 2 minute break 20% of body weight and one rep max single arm dumbbell row for 10 reps for 4 sets each arm 40% of body weight and one rep max for 5 reps for 5 sets 40% of body weight and one rep max dumbbell bench press 8 reps for 2 sets 40% of body weight and one rep max tricep extensions for 8 reps for 2 sets knee push ups for 12 reps for 2 sets 8 knee push ups for 2 sets 15 knee push ups for 2 sets max knee push ups 8 assisted dips for 2 sets 6 dips for 2 sets 10% of body weight and one rep max dumbbell shoulder presses for 15 reps for 2 sets 10% of body weight and one rep max dumbbell front raise for 12 reps for 2 sets 10% of body weight and one rep max dumbbell front raise for 8 reps for 2 sets 10% of body weight and one rep max dumbbell lateral raise for 12 reps for 2 sets 10% of body weight and one rep max dumbbell lateral raise for 8 reps for 2 sets 10% of body weight and one rep max super set for 10 reps for 3 sets dumbbell front raise to lateral raise to shoulder press for 10 reps for 3 sets 40% of body weight and one rep max bench press till failure 5 assisted chin ups for 3 sets once you can do it do 5 assisted chin ups for 3 sets then 10 assisted chin ups for 3 sets so do original 5 assisted chin ups for 3 sets plus 10 assisted chin ups for 3 sets 5 assisted pull ups for 2 sets once you can do 5 assisted pull ups for 2 sets do 5 assisted pull ups for 2 sets then 10 assisted pull ups for 2 sets

### Leg Day

50% of body weight and one rep max average barbell lunges for 6 reps for 3 sets each leg 50% of body weight and one rep max average deadlift for 12 reps for 2 sets 50% of body weight and one rep max average deadlift 8 reps for 2 sets 50% of body weight and one rep max average 6 reps for 2 sets 50% of body weight and one rep max average 4 reps for 4 sets 50% of body weight and one rep max average barbell squat for 15 reps 50% of body weight and one rep max average barbell squat for 10 reps for 2 sets 50% of body weight and one rep max average barbell squats for 8 reps for 2 sets 50% of body weight and one rep max average barbell squats for 6 reps for 2 sets 50% of body weight and one rep max average barbell squats for 5 reps for 2 sets 50% of body weight and one rep max average barbell squats till failure 50% of body weight and one rep max average barbell hip thrusts for 12 reps 50% of body weight and one rep max average hip thrusts 10 reps for 1 set 50% of body weight and one rep max average hip thrusts 8 reps 50% of body weight and one rep max average 6 reps single leg squats 8 reps for 1 set each leg single leg squats 6 reps each leg sets Nordic curls for 12 reps for 1 set Nordic curls for 8 reps for 1 set Nordic curls for 6 reps for 1 sets 50% of body weight and one rep max average leg curls for 12 reps for 2 sets 50% of body weight and one rep max average leg curls till failure wall sit no weight one minute for 2 sets

### Back Day

20 back extensions without weight 20% of body weight and one rep max average 20 back extensions 20 back extensions without weight 20% of body weight and one rep max average 20 back extensions 10 back extensions without weight 20% of body weight and one rep max average 10 back extensions 20% of body weight and one rep max average single arm dumbbell row for 10 reps for 4 sets each arm 20% of body weight and one rep max average lat pull downs for 12 reps 20% of body weight and one rep max average lat pull downs for 8 reps 20% of body weight and one rep max average for 6 reps 20% of body weight and one rep max average close chin up grip lat pull downs for 12 reps 20% of body weight and one rep max average close chin up grip lag pull downs for 8 reps 20% of body weight and one rep max average good mornings 12 reps 20% of body weight and one rep max average good mornings 8 reps 20% of body weight and one rep max average good mornings 6 reps 20% of body weight and one rep max average rack pulls for 12 reps 20% of body weight and one rep max average rack pulls for 8 reps 20% of body weight and one rep max average rack pulls for 6 reps for 2 sets 20% of body weight and one rep max average rack pulls for 4 reps 20% of body weight and one rep max average barbell rows for 12 reps 20% of body weight and one rep max average barbell rows for 8 reps 20% of body weight and one rep max average barbell rows for 6 reps 20% of body weight and one rep max average lat pull downs for 12 reps 20% of body weight and one rep max average lat pull downs for 8 reps 20% of body weight and one rep max average lat pull downs for 6 reps 5 assisted chin ups for once you can do 5 assisted chin ups then do 5 chin ups for 2 sets

15 second reach down touch toes 15 second Chinese splitz hold 15 second reach right leg half split position 15 second reach left leg half split position 15 second reach down middle split position 15 second splitz 15 second sitting half split position right leg 15 second sitting half split position left leg 15 seconds reach forward touch toes 15 second butterfly 15 second hip stretch right leg 15 second hip stretch left leg 15 second knee to chest right leg 15 second knee to chest left leg 15 second flamingo left leg 15 second flamingo right leg 10 leg swings right leg 10 leg swings left leg 20 second knee swivel left 20 second knee swivel right 20 second hip swivel left 20 second hip swivel right 20 second wrist swivel left 20 second wrist swivel right each hand 20 second elbow swivel right 20 second elbow swivel left each arm 20 second shoulder swivel left 20 second shoulder swivel right each arm back to floor on knee stretch 20 seconds 15 seconds arm behind back right arm 15 seconds arm behind back left arm 15 second leg on chair hamstring stretch left leg 15 second leg on chair hamstring stretch right leg 15 second worlds greatest left side 15 second worlds greatest right side 15 seconds leg to side on chair left leg 15 seconds leg side on chair right leg leg swings 10 reps left leg leg swings 10 reps right leg single leg rdl 10 reps left leg single leg rdl 10 reps right leg Warrior 1 15 seconds left leg Warrior 1 15 seconds right leg warrior 2 15 seconds left leg Warrior 2 15 seconds right leg balance on one leg hands forward body leaning forward yoga position 15 seconds left leg balance on one leg hands forward body leaning forward yoga position 15 seconds right leg 15 seconds hip floor wipers 15 second frog hip stretch 15 second Chinese monk squat 20 second ankle rotation left 20 second ankle rotation right each ankle alphabet each ankle 10 second plantar flex 10 second dorsi flex with ankle weight each ankle same thing without weight repeat 10 times calf stretch 10 seconds each leg for 5 sets 10 sets 10 reps calf raises leg extension with weight for 45 second hold for 10 reps same without weight leg extension hold 10 seconds for 10 reps for 5 sets repeat for 1 hour except ankle exercises

One rep max week last month of the year of program when you go for one rep max go 10% above normal after working set after last of working sets 1-3 reps only since going for one rep max till you get to 100% of your one rep max then go for your new one rep max however many sets it takes go as feel comfortable until you can max out only 1 rep

### For Ultra Version of Athlete Program

### Ashwaganda

250-1,000 mg daily 3 capsules daily 0.5 grams of carbs at most

recommend take in morning evening any time or evening about 30 minutes to a hour after meal or before night 30 minutes to a hour before bed and some recommendations 30 minutes to at most two hours before bed

### Tongkat Ali

100-300 mg daily 1 capsule daily 0.30 grams of carbs at most

recommend take in morning 7-8 in morning or between meals or a hour before a workout or super hard workout it can be taken any time of day although it is a natural steroid and testosterone boosting supplement it can also significantly improve energy so can be used as a great pre workout

Tribulus Terrestris Amount 250-1,500 mg daily 2 capsules daily 0 grams of carbs

recommend take 30 minutes after meal empty stomach some recommended take with water 8-12 ounces some recommended take Tribulus before workout for its anabolic effects

### Fadogia Agrestis

100-600 mg daily 1 capsule daily 0 grams of carbs

recommend take with or without meal any time of day

### Omega 3 Fish Oil

1,000-3,000 mg daily 3 capsules daily 0 grams of carbs

recommend take in the morning recommend take at night recommended take with meal but can also take without also recommended take right before meal you can take any time of day

### Pure Creatine

5,000 mg daily 1 gram of carbs recommend take after workout

recommend take before workout

recommend take in morning

recommend take with meal

Georges St Pierre Warrior Liver Supplement 6 capsules daily 0 grams of carbs

recommend take with meal

recommend take after workout

recommended take before workout also

Grass Fed Beef Liver Capsules 6 capsules daily 0 grams of carbs

recommend take with meal

recommend take in the morning

recommend take after workout

recommended take before workout

### Chemix

1 scoop daily 0.20 grams of carbs at very most 2 scoop serving=6,000 mg

recommend take 20-30 minutes before workout

### Gorilla Mind Gorilla Mode

1 scoop daily 0.40 grams of carbs at very most

recommend take 30 minutes to a hour before workout

recommend take right before workout

10-30 minute walking backwards treadmill/no treadmill or with weight or without weight sled every workout day

Do same amount of time doing same movement forward

Back extension hold 5 times every workout day 10 second hold each one

Reverse Nordic curls 12 reps for 2 sets each leg 6 reps for 2 sets each leg every workout day

### For Fighter Program

Also enter Vo2 max and 5 mile run time optional 1 mile rower time max speed and power rower time how long you can hold top speed and power optional 5 mile stationary bike/assault bike time max speed and power stationary bike/assault bike how long you can hold top speed and power optional 1 mile vertimax time max speed and power vertimax how long you can hold top speed and power optional max speed and power jump rope how long you can hold top speed and power

80% of aerobic training in zone 2 and 20% in zone 5 particularly something you can do for 3-8 minutes calculator will use formula above to calculate what you can do for 3-8 minutes with a tiny bit left in tank but not absolutely barely make it just a little left in tank is scientific perfect zone to be in so 3 times a week zone 2 one time a week about something you can do for 4 minutes then 4 minute recover do that 4 times

Exercises rower what can do for 4 minutes about 10% left in Gas tank rower max power for about 4 minutes mile Tun same thing 4 different exercises 4 minutes 4 sets and 3 cardio type days where it is zone 2

### Ultra Version Fighter Program

Ashwaganda Tongkat Ali Tribulus Fabiola Agrestis Fish Oil Pure Creatine Chemix 1st Phorm Hybrid Athlete Stack Prime Sports Nutrition Nutranio Beta Alinine CarnoSyn Beta Alinine Endurance Powder

### Ashwaganda

250-1,000 mg daily 3 capsules daily 0.5 grams of carbs at most

recommend take in morning evening any time or evening about 30 minutes to a hour after meal or before night 30 minutes to a hour before bed and some recommendations 30 minutes to at most two hours before bed

### Tongkat Ali

100-300 mg daily 1 capsule daily 0.30 grams of carbs at most

recommend take in morning 7-8 in morning or between meals or a hour before a workout or super hard workout it can be taken any time of day although it is a natural steroid and testosterone boosting supplement it can also significantly improve energy so can be used as a great pre workout

Tribulus Terrestris Amount 250-1,500 mg daily 2 capsules daily 0 grams of carbs recommend take 30 minutes after meal empty stomach some recommended take with water 8-12 ounces some recommended take Tribulus before workout for its anabolic effects

### Fadogia Agrestis

100-600 mg daily 1 capsule daily 0 grams of carbs

recommend take with or without meal any time of day

Omega 3 fish oil

1,000-3,000 mg daily 3 capsules daily 0 grams of carbs

recommend take in the morning recommend take at night recommended take with meal but can also take without also recommended take right before meal you can take any time of day

### Pure Creatine

5,000 mg daily 1 gram of carbs recommend take after workout

recommend take before workout

recommend take in morning

recommend take with meal

### Chemix

1 scoop daily 0.20 grams of carbs at very most 2 scoop serving=6,000 mg

recommend take 20-30 minutes before workout

1st Phorm Hybrid Athlete Stack 1 packet daily

### Example 3: Male, High end athlete

### Athlete Program

10 minutes direct sunlight after waking up

30 minutes to a hour mentally challenging thing in morning

3 minute cold plunge after

1 hour stretching

15 second reach down touch toes 15 second Chinese splitz hold 15 second reach right leg half split position 15 second reach left leg half split position 15 second reach down middle split position 15 second splitz 15 second sitting half split position right leg 15 second sitting half split position left leg 15 seconds reach forward touch toes 15 second butterfly 15 second hip stretch right leg 15 second hip stretch left leg 15 second knee to chest right leg 15 second knee to chest left leg 15 second flamingo left leg 15 second flamingo right leg 10 leg swings right leg 10 leg swings left leg 20 second knee swivel left 20 second knee swivel right 20 second hip swivel left 20 second hip swivel right 20 second wrist swivel left 20 second wrist swivel right each hand 20 second elbow swivel right 20 second elbow swivel left each arm 20 second shoulder swivel left 20 second shoulder swivel right each arm back to floor on knee stretch 20 seconds 15 seconds arm behind back right arm 15 seconds arm behind back left arm 15 second leg on chair hamstring stretch left leg 15 second leg on chair hamstring stretch right leg 15 second worlds greatest left side 15 second worlds greatest right side 15 seconds leg to side on chair left leg 15 seconds leg side on chair right leg leg swings 10 reps left leg leg swings 10 reps right leg single leg rdl 10 reps left leg single leg rdl 10 reps right leg Warrior 1 15 seconds left leg Warrior 1 15 seconds right leg warrior 2 15 seconds left leg Warrior 2 15 seconds right leg balance on one leg hands forward body leaning forward yoga position 15 seconds left leg balance on one leg hands forward body leaning forward yoga position 15 seconds right leg 15 seconds hip floor wipers 15 second frog hip stretch 15 second Chinese monk squat 20 second ankle rotation left 20 second ankle rotation right each ankle alphabet each ankle 10 second plantar flex 10 second dorsi flex with ankle weight each ankle same thing without weight repeat 10 times calf stretch 10 seconds each leg for 5 sets 10 sets 10 reps calf raises leg extension with weight for 45 second hold for 10 reps same without weight leg extension hold 10 seconds for 10 reps for 5 sets repeat for 1 hour except ankle exercises repeat two times full stretch routine 30 20 second break between every stretch main stretch routine 10 second break between each stretch ankle routine

### 30 Minute Ankle Exercises

Recovery 1 hour a day 10 second each part of calf for 5 sets each leg same for 4 sides a calf 10 seconds for 5 sets and do massage gun for 1 hour or and cryotherapy or thermatech

Ice bath way before workout to not decrease hypertrophy

Sauna after workout to increase hypertrophy

At least 11 minute cold exposure each week

challenge body and maximal strength/joint strength and maximal strength each week do extremely hard movement/movements one rep max or push yourself in position body can't push so it's just pushing against resistance like a wall or so

challenge body and maximal strength/joint strength and maximal strength each week do extremely hard movement/movements one rep max or push yourself in position body can't push so it's just pushing against resistance like a wall or so

Sprint 1-2 times a week

Have to choose at least one 10 minute break in each workout you get to choose you get between 1-3 10 minute breaks per workout except for in stretching no 10 minute break on explosiveness unless program says

Reduce artificial dopamine

### Monk Mode

Mentally challenge each day

Caffeine 90-120 minutes after wake up

Caffeine if you want after or around evening or after Mentally exhausting thing

Reduce light very dark about a hour or so before bed

### Upper Body Day

10% of body weight and one rep max average curls or 10 pound dumbbells minimum for 10 reps for 2 sets each arm 10% of body weight and one rep max average barbell curls 12 reps for 2 sets 10% of body weight and one rep max average Hammer curls 10 reps for 2 sets each arm 40% of body weight and one rep max bench press 20 reps for 5 sets 40% of body weight and one rep max bench press 12 reps 40% of body weight and one rep max 8 reps for 2 sets 40% of body weight and one rep max 6 reps for 2 sets 80-85% of one rep max for 10 reps for 10 sets 2 minute break 20% of body weight and one rep max single arm dumbbell row for 10 reps for 4 sets each arm 40% of body weight and one rep max for 5 reps for 5 sets 40% of body weight and one rep max dumbbell bench press 8 reps for 2 sets 40% of body weight and one rep max tricep extensions for 8 reps for 2 sets Diamond Push Ups for 12 reps for 2 sets 8 finger tip push ups for 2 sets 15 push ups for 2 sets max push ups 8 dips for 2 sets 6 dips for 2 sets 10% of body weight and one rep max dumbbell shoulder presses for 15 reps for 2 sets 10% of body weight and one rep max dumbbell front raise for 12 reps for 2 sets 10% of body weight and one rep max dumbbell front raise for 8 reps for 2 sets 10% of body weight and one rep max dumbbell lateral raise for 12 reps for 2 sets 10% of body weight and one rep max dumbbell lateral raise for 8 reps for 2 sets 10% of body weight and one rep max super set for 10 reps for 3 sets dumbbell front raise to lateral raise to shoulder press for 10 reps for 3 sets 40% of body weight and one rep max bench press till failure 5 chin ups for 3 sets once you can do it do 5 chin ups for 3 sets then 10 chin ups for 3 sets so do original 5 chin ups for 3 sets plus 10 chin ups for 3 sets 5 pull ups for 2 sets once you can do 5 pull ups for 2 sets do 5 pull ups for 2 sets then 10 pull ups for 2 sets

### Leg Day

50% of body weight and one rep max average barbell lunges for 6 reps for 3 sets each leg 50% of body weight and one rep max average deadlift for 12 reps for 2 sets 50% of body weight and one rep max average deadlift 8 reps for 2 sets 50% of body weight and one rep max average 6 reps for 2 sets 50% of body weight and one rep max average 4 reps for 4 sets 50% of body weight and one rep max average barbell squat for 15 reps 50% of body weight and one rep max average barbell squat for 20 reps 50% of body weight and one rep max average barbell squat for 10 reps for 2 sets 50% of body weight and one rep max average barbell squats for 8 reps for 2 sets 50% of body weight and one rep max average barbell squats for 6 reps for 2 sets 50% of body weight and one rep max average barbell squats for 5 reps for 5 sets 50% of body weight and one rep max average barbell squats till failure 50% of body weight and one rep max average barbell hip thrusts for 12 reps 50% of body weight and one rep max average hip thrusts 10 reps for 3 sets 50% of body weight and one rep max average hip thrusts 8 reps 50% of body weight and one rep max average 6 reps 50% of body weight and one rep max average 4 reps bosu ball single leg squats for 6 reps for 2 sets each leg single leg squats 10 reps for 3 sets each leg single leg squats 6 reps each leg 50% of body weight and one rep max barbell box jumps for 5 reps for 4 sets Nordic curls for 12 reps for 3 sets Nordic curls for 8 reps for 3 sets Nordic curls for 6 reps for 2 sets 50% of body weight and one rep max average single leg leg curls for 12 reps for 2 sets 50% of body weight and one rep max average single leg leg curls for 10 reps for 2 sets 50% of body weight and one rep max average leg curls for 12 reps for 2 sets 50% of body weight and one rep max average leg curls till failure 50% of body weight and one rep max average leg curls for 10 reps for 2 sets 50% of body weight and one rep max average leg curls till failure 50% of body weight and one rep max average leg curls for 8 reps for 2 sets 50% of body weight and one rep max average leg curls till failure 50% of body weight and one rep max average squat jumps for 12 reps for 2 sets 50% of body weight and one rep max average squat jumps for 8 reps for 2 sets 50% of body weight and one rep max average squat jumps for 10 reps for 3 sets wall sit with 25 pounds weight for one minute wall sit with 25 pounds weight for 2 minutes wall sit no weight one minute wall sit no weight 2 minutes for 2 sets

15 second reach down touch toes 15 second Chinese splitz hold 15 second reach right leg half split position 15 second reach left leg half split position 15 second reach down middle split position 15 second splitz 15 second sitting half split position right leg 15 second sitting half split position left leg 15 seconds reach forward touch toes 15 second butterfly 15 second hip stretch right leg 15 second hip stretch left leg 15 second knee to chest right leg 15 second knee to chest left leg 15 second flamingo left leg 15 second flamingo right leg 10 leg swings right leg 10 leg swings left leg 20 second knee swivel left 20 second knee swivel right 20 second hip swivel left 20 second hip swivel right 20 second wrist swivel left 20 second wrist swivel right each hand 20 second elbow swivel right 20 second elbow swivel left each arm 20 second shoulder swivel left 20 second shoulder swivel right each arm back to floor on knee stretch 20 seconds 15 seconds arm behind back right arm 15 seconds arm behind back left arm 15 second leg on chair hamstring stretch left leg 15 second leg on chair hamstring stretch right leg 15 second worlds greatest left side 15 second worlds greatest right side 15 seconds leg to side on chair left leg 15 seconds leg side on chair right leg leg swings 10 reps left leg leg swings 10 reps right leg single leg rdl 10 reps left leg single leg rdl 10 reps right leg Warrior 1 15 seconds left leg Warrior 1 15 seconds right leg warrior 2 15 seconds left leg Warrior 2 15 seconds right leg balance on one leg hands forward body leaning forward yoga position 15 seconds left leg balance on one leg hands forward body leaning forward yoga position 15 seconds right leg 15 seconds hip floor wipers 15 second frog hip stretch 15 second Chinese monk squat 20 second ankle rotation left 20 second ankle rotation right each ankle alphabet each ankle 10 second plantar flex 10 second dorsi flex with ankle weight each ankle same thing without weight repeat 10 times calf stretch 10 seconds each leg for 5 sets 10 sets 10 reps calf raises leg extension with weight for 45 second hold for 10 reps same without weight leg extension hold 10 seconds for 10 reps for 5 sets repeat for 1 hour except ankle exercises

### Explosiveness Day

Go to Performance training center Armed Sports/Athletic Republic

### Explosiveness Day

10 minute jump rope 10 single leg box jumps each leg for 2 sets 8 single leg box jumps each leg for 2 sets 6 single leg box jumps each leg for 2 sets 12 box jumps for 2 sets 10 box jumps for 2 sets 6 box jumps for 6 sets 6 box jumps for 4 sets 4 box jumps for 4 sets 50% of body weight and one rep max barbell/dumbbell box jumps 8 reps for 2 sets 50% of body weight and one rep max barbell/dumbbell box jumps for 6 reps for 2 sets 50% of body weight and one rep max barbell/dumbbell box jumps for 4 reps for 2 sets knee jumps for 10 reps for 3 sets bosu ball single leg squats for 6 reps for 2 sets each leg single leg squats 10 reps for 3 sets each leg single leg squats 6 reps each leg bosu ball single leg rdl with 10 pound weight for 8 reps for 2 sets each leg single leg rdl for 8 reps for 2 sets each leg single leg rdl for 6 reps 50% of body weight and one rep max average hip thrusts 10 reps for 3 sets 50% of body weight and one rep max average hip thrusts 8 reps 50% of body weight and one rep max average 6 reps single leg hip thrusts for 10 reps for 3 sets each leg Nordic curls for 12 reps Nordic curls for 8 reps for 2 sets Nordic curls for 6 reps for 2 sets 2 minute walking sideways lateral shuffle both side no weight 2 minute walking side ways lateral shuffle both side 10 pounds weight 50% of body weight and one rep max hang cleans 10 reps 50% of body weight and one rep max hang cleans 8 reps 50% of body weight and one rep max hang cleans 6 reps for 2 sets 50% of body weight and one rep max hang cleans 4 reps for 2 sets 50% of body weight and one rep max barbell squat jumps 10 reps right after 10 reps body weight squat jumps do that for 3 sets 50% of body weight and one rep max calf raises 12 reps for 3 sets 10 50 meter sprints 5 minute break after each sprint finish with 100 m sprint

### Vertical Jump Day

10 single leg box jumps with 10 pound weight each leg 10 single leg box jumps without weight each leg 8 single leg box jumps with 10 pounds weight each leg 8 single leg box jumps without weight each leg 6 single leg box jumps with 10 pound weight each leg 6 single leg box jumps without weight each leg sets 10 minute jump rope 12 box jumps for 2 sets 10 box jumps for 2 sets 6 box jumps for 6 sets 6 box jumps for 4 sets 4 box jumps for 4 sets 50% of body weight and one rep max barbell/dumbbell box jumps 8 reps for 2 sets 50% of body weight and one rep max barbell/dumbbell box jumps for 6 reps for 2 sets 50% of body weight and one rep max barbell/dumbbell box jumps for 4 reps for 2 sets 10 knee jumps for 3 sets 50% of body weight and one rep max barbell squat jumps 10 reps right after 10 reps body weight squat jumps do that for 3 sets 50% of body weight and one rep max calf raises 12 reps for 3 sets

### Back Day

50 back extensions without weight 20 back extensions without weight for 2 sets 20% of body weight and one rep max average 50 back extensions 20% of body weight and one rep max average 20 back extensions 20 back extensions without weight 20% of body weight and one rep max average 20 back extensions 10 back extensions without weight 20% of body weight and one rep max average 10 back extensions for 3 sets 20% of body weight and one rep max average single arm dumbbell row for 10 reps for 4 sets each arm 20% of body weight and one rep max average lat pull downs for 12 reps for 2 sets 20% of body weight and one rep max average lat pull downs for 8 reps for 2 sets 20% of body weight and one rep max average for 6 reps for 2 sets 20% of body weight and one rep max average close chin up grip lat pull downs for 12 reps for 2 sets 20% of body weight and one rep max average close chin up grip lag pull downs for 8 reps for 2 sets 20% of body weight and one rep max average good mornings 12 reps for 2 sets 20% of body weight and one rep max average good mornings 8 reps for 2 sets 20% of body weight and one rep max average good mornings 6 reps for 2 sets 20% of body weight and one rep max average rack pulls for 12 reps for 2 sets 20% of body weight and one rep max average rack pulls for 8 reps for 2 sets 20% of body weight and one rep max average rack pulls for 6 reps for 4 sets 20% of body weight and one rep max average rack pulls for 4 reps for 2 sets 20% of body weight and one rep max average barbell rows for 12 reps for 2 sets 20% of body weight and one rep max average barbell rows for 8 reps for 2 sets 20% of body weight and one rep max average barbell rows for 6 reps for 2 sets 20% of body weight and one rep max average lay pull downs for 12 reps for 2 sets 20% of body weight and one rep max average lay pull downs for 8 reps for 2 sets 20% of body weight and one rep max average lay pull downs for 6 reps for 2 sets 5 chin ups for 2 sets once you can do 5 chin ups for 2 sets then do 5 chin ups for 2 sets plus additional 10 pull ups for 2 sets

### Ab Day

20 leg raises for 5 sets 50 sit ups for 4 sets 20 sit ups for 5 sets decline medicine ball throw sit ups with 11 pound medicine ball for 12 reps for 4 sets 1 minute plank for 2 sets sit ups with 25 pound weight for 12 reps for 2 sets side to side sit ups with 10 pounds weight for 10 reps for 4 sets 50 russian twists with 10 pounds weight for 4 sets 50 russian twists without weight for 4 sets 1 minute plank for 1 set 2 minute plank for 1 set balance on yoga ball for 1 minute for 3 sets flutter kicks for 30 seconds then 6 inch hold for 30 seconds for 5 sets 20 hanging knee/leg raise if can't do leg raise knee raise for 20 reps for 2 sets 10 hanging leg raises for 5 sets 30 second leg Ab hold for 2 sets 1 minute plank for 3 sets each side v ups 20 reps for 5 sets v ups 10 reps for 5 sets get punched in the stomach 20 seconds for 3 sets

The present disclosure has described particular implementations having various features. In light of the disclosure provided above, it will be apparent to those skilled in the art that various modifications and variations can be made without departing from the scope or spirit of the disclosure. One skilled in the art will recognize that the disclosed features may be used singularly, in any combination, or omitted based on the requirements and specifications of a given application or design. When an implementation refers to “comprising” certain features, it is to be understood that the implementations can alternatively “consist of” or “consist essentially of” any one or more of the features. Other implementations will be apparent to those skilled in the art from consideration of the specification and practice of the disclosure.

It is noted in particular that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the disclosure fall within the scope of the disclosure. Further, all of the references cited in this disclosure including patents, published applications, and non-patent literature are each individually incorporated by reference herein in their entireties and as such are intended to provide an efficient way of supplementing the enabling disclosure as well as provide background detailing the level of ordinary skill in the art.

1. A computer-implemented comprising:
inputting one or more parameters of a user on a user interface by a user; and
scheduling, monitoring, tracking, and/or suggesting or recommending components of exercise and health regimens for the user based on the inputting of the one or more parameters;
wherein the scheduling, monitoring, tracking, and/or suggesting or recommending components of exercise and health regimens are specified to the user inputting the one or more parameters;
wherein the one or more parameters comprise user-specific parameters chosen from height, weight, age, gender at birth, body fat percentage, waist size, hip size, shoulder size, chest size, neck circumference, bicep circumference, leg length, and arm length;
wherein the components of exercise regimens are chosen from a particular exercise equipment or method, muscles targeted, speed, power, length of exercise, weight or resistance, number of repetitions, number of sets, length of breaks between sets or activities, and workout schedule;
wherein the components of health regimens are not components of exercise regimens.

2. The computer-implemented method of claim 1, wherein the scheduling comprises displaying a calendar that populates dates and times with the components of the exercise and health regimens.

3. The computer-implemented method of claim 1, wherein the monitoring and tracking comprises input or output that tracks or monitors achievements of various health or fitness goals.

4. The computer-implemented method of claim 1, wherein the suggesting or recommending comprises output of suggested workouts or programs that can be implemented by the user.

5. The computer-implemented method of claim 1, wherein the components of health regimens are chosen from nutritional information, supplement information, activities, mental challenges, and sports.

6. One or more non-transitory, computer-readable storage media having instructions for execution by one or more processors, the instructions programmed to cause the one or more processors to:
input one or more parameters on a user on a user interface by a user; and
schedule, monitor, track, and/or suggest or recommend components of exercise and health regimens for the user based on the inputting of the one or more parameters;
wherein the scheduling, monitoring, tracking, and/or suggesting or recommending components of exercise and health regimens are specified to the user inputting the one or more parameters;
wherein the one or more parameters comprise user-specific parameters chosen from height, weight, age, gender at birth, body fat percentage, waist size, hip size, shoulder size, chest size, neck circumference, bicep circumference, leg length, and arm length;
wherein the components of exercise regimens are chosen from a particular exercise equipment or method, muscles targeted, speed, power, length of exercise, weight or resistance, number of repetitions, number of sets, length of breaks between sets or activities, and workout schedule; and
wherein the components of health regimens are not components of exercise regimens.

7. The one or more non-transitory, computer-readable storage media of claim 6, wherein the scheduling comprises displaying a calendar that populates dates and times with the components of the exercise and health regimens.

8. The one or more non-transitory, computer-readable storage media of claim 6, wherein the monitoring and tracking comprises input or output that tracks or monitors achievements of various health or fitness goals.

9. The one or more non-transitory, computer-readable storage media of claim 6, wherein the suggesting or recommending comprises output of suggested workouts or programs that can be implemented by the user.

10. The, one or more non-transitory, computer-readable storage media of claim 6, wherein the components of health regimens are chosen from nutritional information, supplement information, activities, mental challenges, and sports.

11. A computer or computer system, comprising:
one or more processors designed to execute instructions; and
one or more non-transitory, computer-readable memories storing program instructions for execution by the one or more processors, the instructions programmed to cause the one or more processors to:
input one or more parameters on a user on a user interface by a user; and
schedule, monitor, track, and/or suggest or recommend components of exercise and health regimens for the user based on the inputting of the one or more parameters;
wherein the scheduling, monitoring, tracking, and/or suggesting or recommending components of exercise and health regimens are specified to the user inputting the one or more parameters;
wherein the one or more parameters comprise user-specific parameters chosen from height, weight, age, gender at birth, body fat percentage, waist size, hip size, shoulder size, chest size, neck circumference, bicep circumference, leg length, and arm length;
wherein the components of exercise regimens are chosen from a particular exercise equipment or method, muscles targeted, speed, power, length of exercise, weight or resistance, number of repetitions, number of sets, length of breaks between sets or activities, and workout schedule; and
wherein the components of health regimens are not components of exercise regimens.

12. The computer or computer system of claim 11, wherein the scheduling comprises displaying a calendar that populates dates and times with the components of the exercise and health regimens.

13. The computer or computer system of claim 11, wherein the monitoring and tracking comprises input or output that tracks or monitors achievements of various health or fitness goal

14. The computer or computer system of claim 11, wherein the suggesting or recommending comprises output of suggested workouts or programs that can be implemented by the user.

15. The computer or computer system of claim 11, wherein the components of health regimens are chosen from nutritional information, supplement information, activities, mental challenges, and sports.
